(19) United States
(12) Patent Application Publication
(10) Pub. No.: US 2024/0221955 A1
(43) Pub. Date: Jul. 4, 2024

(54) PROSPECTIVE CLASSIFICATION DEVICE FOR PREDICTING DEMENTIA AND OPERATION METHOD OF THE SAME

(71) Applicant: AJOU UNIVERSITY INDUSTRY-ACADEMIC COOPERATION FOUNDATION, Suwon-si (KR)

(72) Inventors: Hyunjung Shin, Suwon-si (KR); Chang Hyung Hong, Seongnam-si (KR); Sang Joon Son, Seoul (KR); Hyun Woong Roh, Suwon-si (KR); Sunghong Park, Suwon-si (KR)

(73) Assignee: AJOU UNIVERSITY INDUSTRY-ACADEMIC COOPERATION FOUNDATION, Suwon-si (KR)

(21) Appl. No.: 18/401,742

(22) Filed: Jan. 2, 2024

(30) Foreign Application Priority Data
Dec. 30, 2022 (KR) 10-2022-0190872

(51) Int. Cl. G16H 50/30; A61B 5/00; G16H 30/40

(52) U.S. Cl. CPC G16H 50/30; A61B 5/4088; G16H 30/40

(57) ABSTRACT

A prospective classification device for predicting dementia that predicts a risk of a patient with mild cognitive impairment being converted to a dementia patient based on the characteristics of prognostic brain imaging data converted from a diagnostic brain imaging data and a method of operating the same are disclosed. The prospective classification device is configured to convert features of the diagnostic brain imaging data obtained at the time of diagnosis of a patient with mild cognitive impairment into features of prognostic brain imaging data corresponding to the prognostic time after the time of diagnosis using a prospective classification model.

### STATEMENT REGARDING GOVERNMENT SPONSORED RESEARCH OR DEVELOPMENT

The present disclosure was developed in the task of a project to develop an artificial intelligence model for dementia precision medicine and diversification of diagnosis (Project identification number: 1711188850, Project number: 2021R1A2C2003474, Ministry name: Ministry of Science and ICT, Project management organization name: National Research Foundation of Korea, Research project name: Individual basic research (Ministry of Science and ICT), contribution rate: 10/100, project implementation organization name: Ajou University, research period: 2023.03.01-2024.02.29.)

### CROSS-REFERENCE TO RELATED APPLICATIONS

This application claims priority under 35 U.S.C. § 119 to Korean Patent Application No. 10-2022-0190872 filed on Dec. 30, 2022 in the Korean Intellectual Property Office, the disclosures of which are incorporated by reference herein in their entireties.

The present disclosure was developed in the task of a project of the Ajou DREAM Artificial Intelligence Innovation Talent Training Project (Project Identification Number: 1345360502, Project Number: 5199991014091, Ministry Name: Ministry of Education, Project Management Agency Name: National Research Foundation of Korea, Research Project Name: 4th Stage Brain Korea 21 Project (R&D), Contribution Rate: 10/100, name of project performing organization: Ajou University, research period: 2022.03.01˜2023.02.28.)

The present disclosure was developed in the task of a developing a voice phishing information collection, processing, and big data-based investigation support system (task identification number: 1711200908, task number: 2022-0-00653, ministry name: Ministry of Science and ICT, project management agency name: Information and Communication Planning and Evaluation Institute, This is a technology developed through Research Project Name: Development of Technology to Prevent Illegal Mobile Phone Use, Contribution Rate: 10/100, Project Implementation Agency Name: National Police University, Research Period: 2023.01.01-2023.12.31.)

The present disclosure was developed in the task of the Artificial Intelligence Convergence Innovation Talent Training (Ajou University) Project (Project Identification Number: 1711197986, Project Number: 00255968, Ministry Name: Ministry of Science and ICT, Project Management Agency Name: Information and Communications Planning and Evaluation Institute, Research Project Name: Artificial Intelligence Convergence Innovation Talent This is a technology developed through training, contribution rate: 10/100, project performing organization name: Ajou University Industry-Academic Cooperation Foundation, research period: 2023.07.01˜2023.12.31.)

The present disclosure was developed in the task of a multimodal-multidomain-based machine learning algorithm for predicting dementia progression (Project identification number: 1345371666, Project number: 2022R1A6A3A01086784, Ministry name: Ministry of Education, Project management organization name: National Research Foundation of Korea, Research project name: Science and Engineering Research This is a technology developed through foundation construction, contribution rate: 40/100, project carrying out organization name: Ajou University Industry-Academic Cooperation Foundation, research period: 2023.09.01˜2024.08.31.)

The present disclosure was developed in the task of the Human-Environmental Interaction Beyond Target Platform Construction Project (Task Identification Number: 1465039357, Project Number: HR21C1003020023, Ministry Name: Ministry of Health and Welfare, Project Management Agency Name: Korea Health Industry Development Institute, Research Project Name: Research-oriented Hospital Development, Contribution Rate: 5/100, project carrying out organization name: Ajou University Industry-Academic Cooperation Foundation, research period: 2023.01.01˜2023.12.31.)

The present disclosure was developed in the task of a super-gap SUPER*Senior Total Health Care platform project (task identification number: 1465039698, task number: HR22C1734010023, ministry name: Ministry of Health and Welfare, project management agency name: Korea Health Industry Development Institute, research project name: Research-oriented hospital development, contribution rate: 5 This is a technology developed through/100, project performing organization name: Ajou University Hospital, research period: 2023.01.01-2023.12.31.)

The present disclosure was developed in the task of a brain disease convergence research center project (project identification number: 1711191592, task number: 2019R1A5A2026045, ministry name: Ministry of Science and ICT, project management agency name: National Research Foundation of Korea, research project name: group research support, contribution rate: 10/100, task This is a technology developed through the name of the performing organization: Ajou University, research period: 2023.03.01-2024.02.29.)

The present disclosure was developed in the task of the Innovative Chronic Cerebrovascular Disease Biobank (Detailed Project Number: KBN4-B02-2023-01) support project.

Meanwhile, in all the aspects of the inventive concept, there is no property interest in the government of the Republic of Korea.

### BACKGROUND

The present disclosure relates to a prospective classification device for predicting dementia and a method of operating the same, and more specifically, to a prospective classification device for predicting the transition from mild cognitive impairment to Alzheimer's disease.

Alzheimer's disease (AD) is the most common dementia affecting the elderly people. The number of AD patients continues to increase in line with the rapid aging of the elderly population, and the global AD population is expected to triple from approximately 50 million in 2015 to 131, 5 million in 2050. As such, AD is emerging as a serious problem in recent, but prevention and delay of progression are the only solutions due to the unclear cause of the disease and the absence of a therapy. However, most of the patients will be tested and diagnosed with AD already onset. Therefore, it suggests the importance of predicting disease progression by early detection of the potential risk.

Although current definition tends to describe AD evolution as a continuum of beta-amyloid accumulation, Mild Cognitive Impairment (MCI) is regarded as a prodromal phase of AD based on clinical symptoms. Slight memory loss or other cognitive ability loss suffered by MCI patients is considered an early stage of AD symptoms such as long-term memory loss, language impairment, disorientation, and change in personality. Previous studies have suggested that approximately 12% of subjects suffering from MCI progress to AD in the four years following the first symptoms. Consequently, early diagnosis of the potential risk of AD boils down to the task of predicting MCI patients will convert to AD or not.

MCI and AD are associated with changes observed in brain imaging, particularly loss of brain volume (atrophy) and the development of focal lesions of the white and gray matter. Advances in medical imaging techniques such as magnetic resonance imaging (MRI) have led to the development of many quantitative methods to improve the capabilities of computer-aided systems to facilitate early detection of AD. Moreover, by applying machine learning algorithms, those methods became possible to discover subtle patterns of brain volume loss more precisely. Typically, machine learning applied to brain MRI is to train algorithms on a set of preprocessed features, such as regional volumes and cortical thickness, to create a classifier which predicts the correct diagnostic outcome for new observations.

For the prediction of AD conversion from MCI, existing methods have been conducted using supervised learning techniques that aim at inducing a decision surface from a set of images labeled as MCI-C (MCI conversion) and MCI-N (MCI non-conversion). The most popular classifiers are linear discriminant analysis (LDA), sparse representation classifier (SRC), and support vector machine (SVM). In LDA based methods, they proposed to find a linear combination of features to best separate the two groups. Wolz et al. performed manifold learning with MR images combining multiple features such as hippocampal volume, tensor-based morphometry and cortical thickness. Cho et al. classified AD patients with incremental method using cortical thickness data in terms of spatial frequency components, employing the manifold harmonic transform.

On the other hand, in SRC based methods, they attempted to find a sparse linear combination with minimum representation residual. Xu et al. developed a multi-modality classification framework including three modalities of volumetric MRI, fluorodeoxyglucose and positron emission tomography (PET). Chen et al. proposed a group discriminative sparse representation algorithm with the analysis for the efficiency of the group label information.

Lastly, in SVM based methods, nonlinear decision boundaries were found that maximally separate MCI-C and MCI-N using kernel trick. Zhang et al. proposed 3D discrete wavelet transform based MRI preprocessing method and validated the effectiveness in early diagnosis of ADs and MCIs. Wei et al. distinguished MCI-C and MCI-N by using a combination of FreeSurfer-derived MRI features and nodal features derived from the thickness network. In addition to the above, various machine learning algorithms contribute to deriving more sophisticated tools for predicting AD conversion risk in patients with MCI.

Recently, various methods applying deep learning have been developed, and in particular, prediction results based on Convolutional Neural Networks (CNN) show high accuracy. The convolutional layer, the core of CNN, have the advantage of sharing parameters being locally connected, which helps the network correctly assumes about the nature of images: stationarity of statistics and locality of pixel dependencies. LeNet, AlexNet, VGGNet, and GoogLeNet are considered as representative CNN structures through the success of ImageNet competitions. However, those structures have a critical issue of the vanishing gradient in training a deep model. By solving this issue through shortcut connections allowing input from lower layers of the network to be available at higher layers, ResNet is being used more widely than the aforementioned CNN structures. Furthermore, as the CNN structure has been improved to learn not only flat images but also solid images, it has become easier to accurately predict AD conversion from MCI using brain images themselves.

Despite these successes, predicting AD conversion is still a challenging task due to a couple of reasons with respect to the variability of subject groups. First, MCI-C and MCI-N groups have minor inter-group difference in brain MRI data. As a single category of MCI, both groups can be commonly viewed as an intermediate state between cognitive normal (CN) and AD. Unlike classification of MCI versus CN or AD, it is difficult to distinguish the brain volume differences between MCI-C and MCI-N. On the other hand, the second is that both groups have large intra-group variations. The criteria for MCI are not strict, and the range of subjects is wide, so there is great individual variation in symptoms. Also, the correlation between the severity of MCI and the risk of conversion to AD is not clear. For these reasons, the brain volumes of subjects in the same group can be very different, so high variation within group is inevitable. Therefore, these problems incur difficulties in finding an appropriate classifier to distinguish whether an MCI patient will convert to AD or not.

### SUMMARY

Embodiments of the present disclosure provide a prospective classification device for predicting dementia and a method of operating the same which converts features of diagnostic brain imaging data acquired at the time of diagnosis of a patient with mild cognitive impairment into features of prognostic brain imaging data corresponding to the prognostic time after the time of diagnosis, thereby predicting a risk of a patient with mild cognitive impairment being converted to a dementia patient.

A prospective classification device for predicting dementia according to an embodiment of the present invention comprises at least one processor configured to predict a risk of a mild cognitive impairment patient being converted to a dementia patient by executing a prospective classification program recorded in memory.

The at least one processor is configured to convert features of diagnostic brain imaging data of the patient with mild cognitive impairment obtained at the time of diagnosis into features of prognostic brain imaging data corresponding to a prognostic time after the time of diagnosis by a prospective classification model; and predict the risk of the mild cognitive impairment patient being converted into the dementia patient based on the features of the prognostic brain imaging data converted from the features of the diagnostic brain imaging data.

In one embodiment, the prospective classification model is a model trained to transform features of diagnostic brain imaging data acquired for patients suffering from mild cognitive impairment at a first time point to prognostic brain imaging data acquired at a second time point for the patients after the first time point.

In one embodiment, the at least one processor is configured to convert a diagnostic brain image data matrix obtained at the time of diagnosis of the patient with mild cognitive impairment to generate a projection data matrix by a projection matrix of the trained prospective classification model; smooth the projection data matrix to adapt to a manifold of prognostic brain imaging data matrix to generate a prospective data matrix by a brain graph matrix of the trained prospective classification model; and predict the risk the patient with mild cognitive impairment being converted to a dementia patient by calculating a dementia conversion risk score indicating a probability that mild cognitive impairment being converted to dementia, the dementia conversion risk score being calculated by applying a coefficient vector of the prospective classification model to the prospective data matrix.

In one embodiment, the at least one processor is configured to generate the brain graph matrix based on a correlation matrix representing connection information of feature regions of the prognostic brain image data matrix, and a diagonal matrix of the correlation matrix.

In one embodiment, the at least one processor is configured to convert the diagnostic brain image data matrix of each patient suffering from mild cognitive impairment by a projection matrix to generate a projection data matrix; calculate a brain graph matrix representing a manifold of prognostic brain image data matrix for each of the first patients who suffered from mild cognitive impairment and then converted to dementia and the second patients not convert to dementia after suffering from mild cognitive impairment; generate a prospective data matrix by smoothing the projection data matrix of each of the first patients and the second patients to adapted to the manifold of the prognostic brain image data matrix using the brain graph matrix; generate a divergence function representing a distribution difference between the prospective data matrix generated for each of the first patients and the second patients and the prognostic brain image data matrix of the patient corresponding to each prospective data matrix; calculate a dementia conversion risk score indicating a probability that mild cognitive impairment converts to dementia by variables including the prospective data matrix and coefficient vector for each of the first patients and the second patients; generate a cross-entropy loss function between the dementia conversion risk score calculated for each of the first patients and the second patients and the dementia conversion correct answer labels of the first patients and the second patients; and optimize the projection matrix and the coefficient vector based on a derivative generated by partial differentiation of an objective function defined by the divergence function and the cross-entropy loss function with respect to the projection matrix and the coefficient vector.

In one embodiment, the at least one processor is configured to calculate a first gradient function of the objective function with respect to the projection matrix based on a first derivative of the cross entropy loss function with respect to the projection matrix and a second derivative of the divergence function with respect to the projection matrix; calculate a second gradient function for the coefficient vector of the objective function based on a third derivative of the cross-entropy loss function with respect to the coefficient vector; and optimize the projection matrix and the coefficient vector by deriving an optimal solution of the projection matrix and the coefficient vector in which the magnitudes of the first gradient function and the second gradient function are simultaneously minimized.

In one embodiment, the first gradient function includes the first derivative, the second derivative, and the linear function of the projection matrix, and the second gradient function includes the third derivative and the linear function of the coefficient vector.

In one embodiment, the at least one processor is configured to convert the prospective data matrix and the prognostic brain image data matrix into a prospective probability data matrix and a prognostic brain image probability data matrix, respectively, using a softmax function; and generate a Kullback-Leibler divergence function representing the distribution difference between the prospective probability data matrix and the prognostic brain image probability data matrix.

In one embodiment, the at least one processor is configured to generate a first sub-objective function by applying a first combination coefficient to the cross-entropy loss function; generate a second sub-objective function by applying a second coupling coefficient to the divergence function; generate a normalization term based on a size of the projection matrix and a size of the coefficient vector to reduce the complexity of the projection matrix and the coefficient vector; and generate the objective function based on the first sub-objective function, the second sub-objective function, and the normalization term.

An operation method of prospective classification device for predicting dementia according to an embodiment of the present invention comprises predicting a risk of a mild cognitive impairment patient being converted to a dementia patient by executing a prospective classification program recorded in memory by at least one processor.

In one embodiment, the predicting of the risk comprises: converting features of diagnostic brain imaging data of the patient with mild cognitive impairment obtained at the time of diagnosis into features of prognostic brain imaging data corresponding to a prognostic time after the time of diagnosis by a prospective classification model; and predicting the risk of the mild cognitive impairment patient being converted into the dementia patient based on the features of the prognostic brain imaging data converted from the features of the diagnostic brain imaging data.

In one embodiment, the converting of features of the diagnostic brain imaging data comprises converting a diagnostic brain image data matrix obtained at the time of diagnosis of the patient with mild cognitive impairment to generate a projection data matrix by a projection matrix of the trained prospective classification model; smoothing the projection data matrix to adapt to a manifold of prognostic brain imaging data matrix to generate a prospective data matrix by a brain graph matrix of the trained prospective classification model; and predicting the risk the patient with mild cognitive impairment being converted to a dementia patient by calculating a dementia conversion risk score indicating a probability that mild cognitive impairment being converted to dementia, the dementia conversion risk score being calculated by applying a coefficient vector of the prospective classification model to the prospective data matrix.

In one embodiment, the converting of features of the diagnostic brain imaging data further comprises generating the brain graph matrix based on a correlation matrix representing connection information of feature regions of the prognostic brain image data matrix, and a diagonal matrix of the correlation matrix.

The operation method of prospective classification device for predicting dementia according to an embodiment of the present invention further comprises learning the prospective classification model based on the diagnostic brain imaging data and the prognostic brain imaging data, by the at least one processor.

In one embodiment, the learning of the prospective classification model comprises converting the diagnostic brain image data matrix of each patient suffering from mild cognitive impairment by a projection matrix to generate a projection data matrix; calculating a brain graph matrix representing a manifold of prognostic brain image data matrix for each of the first patients who suffered from mild cognitive impairment and then converted to dementia and the second patients not convert to dementia after suffering from mild cognitive impairment; generating a prospective data matrix by smoothing the projection data matrix of each of the first patients and the second patients to adapted to the manifold of the prognostic brain image data matrix using the brain graph matrix; generating a divergence function representing a distribution difference between the prospective data matrix generated for each of the first patients and the second patients and the prognostic brain image data matrix of the patient corresponding to each prospective data matrix; calculating a dementia conversion risk score indicating a probability that mild cognitive impairment converts to dementia by variables including the prospective data matrix and coefficient vector for each of the first patients and the second patients; generating a cross-entropy loss function between the dementia conversion risk score calculated for each of the first patients and the second patients and the dementia conversion correct answer labels of the first patients and the second patients; and optimizing the projection matrix and the coefficient vector based on a derivative generated by partial differentiation of an objective function defined by the divergence function and the cross-entropy loss function with respect to the projection matrix and the coefficient vector.

In one embodiment, the optimizing of the projection matrix and the coefficient vector comprises calculating a first gradient function of the objective function with respect to the projection matrix based on a first derivative of the cross entropy loss function with respect to the projection matrix and a second derivative of the divergence function with respect to the projection matrix; calculating a second gradient function for the coefficient vector of the objective function based on a third derivative of the cross-entropy loss function with respect to the coefficient vector; and optimizing the projection matrix and the coefficient vector by deriving an optimal solution of the projection matrix and the coefficient vector in which the magnitudes of the first gradient function and the second gradient function are simultaneously minimized.

In one embodiment, the first gradient function includes the first derivative, the second derivative, and the linear function of the projection matrix, and the second gradient function includes the third derivative and the linear function of the coefficient vector.

In one embodiment, the generating of the divergence function comprises converting the prospective data matrix and the prognostic brain image data matrix into a prospective probability data matrix and a prognostic brain image probability data matrix, respectively, using a softmax function; and generating a Kullback-Leibler divergence function representing the distribution difference between the prospective probability data matrix and the prognostic brain image probability data matrix.

In one embodiment, the learning of the prospective classification model comprises generating a first sub-objective function by applying a first combination coefficient to the cross-entropy loss function; generating a second sub-objective function by applying a second coupling coefficient to the divergence function; generating a normalization term based on a size of the projection matrix and a size of the coefficient vector to reduce the complexity of the projection matrix and the coefficient vector; and generating the objective function based on the first sub-objective function, the second sub-objective function, and the normalization term.

In one embodiment of the present invention, a computer-readable non-transitory recording medium is provided on which a computer program for executing an operation method of a prospective classification device for predicting dementia according to the operation method of the prospective classification device is recorded.

### BRIEF DESCRIPTION OF THE FIGURES

The above and other objects and features of the present disclosure will become apparent by describing in detail embodiments thereof with reference to the accompanying drawings.

FIG. 1 is a conceptual diagram of an operation method of a prospective classification device for predicting dementia according to an embodiment of the present invention.

FIG. 2 is a flowchart of a method of operating a prospective classification device for predicting dementia according to an embodiment of the present invention.

FIG. 3 is a conceptual diagram showing the operation of a prospective classification device for predicting dementia according to an embodiment of the present invention.

FIG. 4 is a flowchart specifically showing step S100 of FIG. 2.

FIG. 5 is a flowchart showing the steps of generating a divergence function in step S110 of FIG. 4.

FIG. 6 is a flowchart specifically showing step S140 of FIG. 4.

FIG. 7 is a flowchart specifically showing step S200 and step S300 of FIG. 2.

FIG. 8 is an example diagram of diagnostic data and prognostic data to explain the operation method of the prospective classification device according to an embodiment of the present invention.

FIGS. 9 and 10 are conceptual diagrams showing the process of converting diagnostic brain image data into prognostic brain image data according to an embodiment of the present invention.

FIG. 11 is a conceptual diagram of a prospective classification model according to an embodiment of the present invention.

FIG. 12 is example of Hammers atlas which consists of 95 ROIs.

FIGS. 13 to 15 are results for prospective classification of the present invention.

FIGS. 16 to 18 show comparison results of the present invention.

FIG. 19 shows the results of the present invention for comparison analysis with CNN.

### DETAILED DESCRIPTION

Embodiments described in this specification are intended to clearly describe the spirit of the present disclosure to those skilled in the art to which the present disclosure pertains, and therefore the present disclosure is not limited to the embodiments described in this specification, and the scope of the present disclosure should be construed to include modifications or variations that do not depart from the spirit of the present disclosure.

A prospective classification device for predicting dementia and a method of operating the same according to an embodiment of the present invention transform the features of diagnostic brain imaging data of a patient with mild cognitive impairment acquired at the time of diagnosis into a prognostic point after the time of diagnosis by using a prospective classification model. The present invention predicts a risk of a patient with mild cognitive impairment being converted into a dementia patient using the features of the prognostic brain imaging data converted from the features of the diagnostic brain imaging data. To this end, the prospective classification model is learned (trained) to convert features of diagnostic brain imaging data for patients with mild cognitive impairment obtained at a first time point to features of prognostic brain imaging data for the patients obtained at a second time point after the first time point.

After a certain period of time, MCI-C shows progression to AD due to significant brain atrophy, whereas MCI-N shows only subtle changes due to the normal aging effect. This indicates a difference in brain volume between the two groups at some point in the future.

Motivated by this, the present invention proposes a novel method called prospective classification for predicting the conversion from MCI to AD. The present invention predicts the risk of dementia using a longitudinal transition of how a subject's brain volume changes from the present diagnosis to the future prognosis, using trained prospective classification model.

That is, the current brain image is projected into the future along with the transition pattern and classified where there is a larger difference between groups. The present invention classifies MCI patients into AD conversion group and non-conversion group using projected brain images to the prognosis time rather than using the current image at the time of diagnosis. Patients who progress from mild cognitive impairment to dementia progress to AD show significant brain atrophy over a period of time, whereas patients who do not convert from mild cognitive impairment to dementia show only subtle changes due to normal aging effects. This represents the difference in brain volume between the two groups at some points in the future. Classification may be easier by using longitudinal transitions indicating how a subject's corpus callosum changes from current diagnosis to future prognosis. In other words, the current brain image is projected into the future along with the transition pattern to classify where there is a large difference between groups.

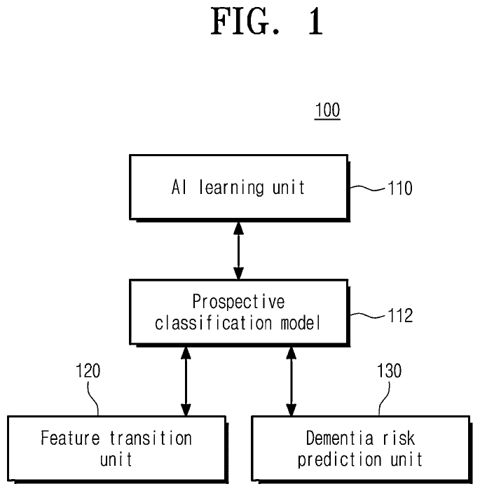

FIG. 1 is a conceptual diagram of an operation method of a prospective classification device for predicting dementia according to an embodiment of the present invention. The operation method of the prospective classification device for dementia prediction according to an embodiment of the present invention determines the pattern of brain transition from diagnosis to prognosis for the MCI to AD conversion group and the MCI to AD non-conversion group, respectively. The classifier is then trained using more distinctly distinguishable transitioned brain volume features and predicts the AD conversion risk of the patients.

The terms used herein are general terms that are currently widely used as much as possible in consideration of their function in the present disclosure, but may vary depending on the intention of a person skilled in the art in the technical field to which the present disclosure pertains, precedents, or the emergence of new technology. When a specific term is defined and used with an arbitrary meaning, the meaning of the term will be described separately. Accordingly, the terms used in the present specification are not to be defined as simple names of the components, but should be defined on the basis of the actual meaning of the terms and the whole context throughout the present specification.

The accompanying drawings are to facilitate the explanation of the present disclosure, and a shape in the drawings may be exaggerated for the purpose of convenience of explanation so the present disclosure is not limited to the drawings. In the present specification, if it is determined that a detailed description of a known configuration or function related to the present disclosure may obscure the gist of the present disclosure, the detailed description thereof will be omitted as necessary.

The operating method of the prospective classification device for dementia prediction according to an embodiment of the present invention trains the brain transition pattern from diagnosis to prognosis for the MCI to AD conversion group and the MCI to AD non-conversion group, respectively (pattern of brain transition). That is, a classifier is trained using the more distinct transitioned brain volume features, and then a person's risk of transitioning to AD is predicted by the trained classifier. The classifier predicts a subject's risk of AD conversion based on the prognostic characteristics of brain volume, which may lead to better performance because the differences between the two groups in prognostic characteristics are more pronounced than at the time of diagnosis.

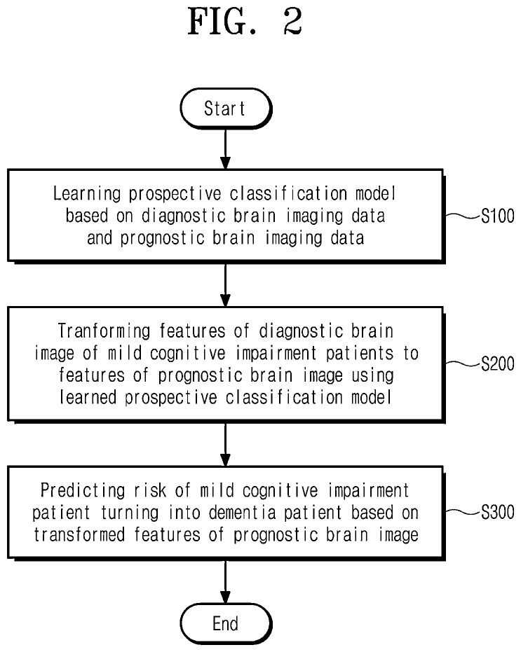

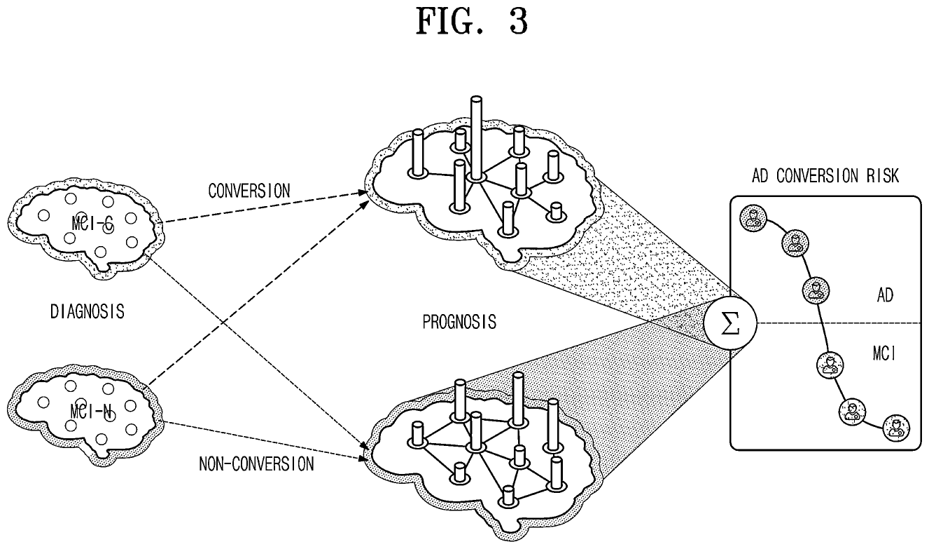

FIG. 1 is a configuration diagram of a prospective classification device for predicting dementia according to an embodiment of the present invention. FIG. 2 is a flowchart of a method of operating a prospective classification device for predicting dementia according to an embodiment of the present invention. FIG. 3 is a conceptual diagram showing the operation of a prospective classification device for predicting dementia according to an embodiment of the present invention. Referring to FIGS. 1 to 3, the prospective classification device 100 for predicting dementia according to an embodiment of the present invention includes an AI (artificial intelligence) learning unit 110, a feature transition unit 120, and a dementia risk prediction unit 130. The AI learning unit 110, feature transition unit 120, and dementia risk prediction unit 130 may perform their functions by having at least one processor execute a prospective classification algorithm (program) stored in memory.

The operation method of a prospective classification device for predicting dementia according to an embodiment of the present invention includes generating a prospective classification model based on diagnostic brain imaging data and prognostic brain imaging data by the AI learning unit 110 (S100); converting, by the feature transition unit 120, the features of the diagnostic brain imaging data of a patient with mild cognitive impairment acquired at the time of diagnosis into features of the prognostic brain imaging data corresponding to the prognostic time after the diagnosis by the learned prospective classification model (S200); and predicting, by the dementia risk prediction unit 130, the risk of a patient with mild cognitive impairment being converted into a dementia patient based on the features of the prognostic brain imaging data converted from the features of the diagnostic brain imaging data (S300).

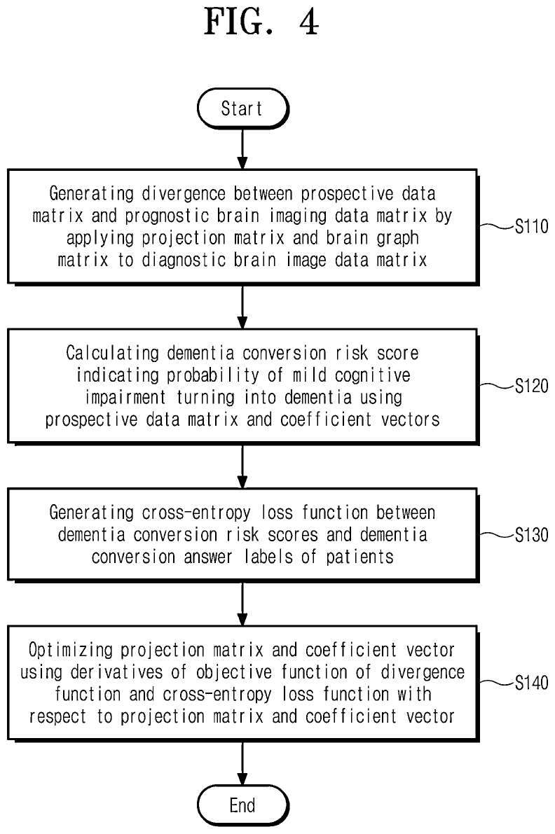

FIG. 4 is a flowchart specifically showing step S100 of FIG. 2. Referring to FIGS. 1, 2, and 4, the step (S100) of learning a prospective classification model includes generating a prospective data matrix by a projection matrix and a brain graph matrix from the diagnostic brain image data matrix of each patient suffering from mild cognitive impairment, and generating a divergence function representing the distribution difference between the prospective data matrix and the patient's prognostic brain image data matrix (S110); calculating a dementia conversion risk score indicating the probability that mild cognitive impairment converts to dementia by variables including a prospective data matrix and coefficient vector for each of the first and second patients (S120); generating a cross-entropy loss function between the dementia conversion risk scores calculated for each of the first and second patients and the dementia conversion correct answer labels of the first and second patients (S130); optimizing the projection matrix and the coefficient vector based on the partial derivative of the objective function defined by the cross entropy loss function and the divergence function with respect to the projection matrix and the coefficient vector (S140).

In step S110, the AI learning unit 110 generates a projection data matrix by converting the diagnostic brain image data matrix of each patient suffering from mild cognitive impairment using a projection matrix to learn a prospective classification model, and generates a projection data matrix. The AI learning unit 110 generates a brain graph matrix representing a manifold of prognostic brain image data matrix for each of the first patients who developed dementia after experiencing mild cognitive impairment and the second patients who did not develop dementia after experiencing mild cognitive impairment.

The AI learning unit 110 generates a prospective data matrix by smoothing the projection data matrix of each of the first patients and the second patients to adapt to the manifold of the prognostic brain image data matrix by the brain graph matrix. The AI learning unit 110 generates a divergence function representing the distribution difference between the prospective data matrix generated for each of the first and second patients and the patient's prognostic brain image data matrix corresponding to each prospective data matrix. At this time, the brain graph matrix may be generated by a correlation matrix representing connection information of feature regions of the prognostic brain image data matrix and the diagonal matrix of the correlation matrix.

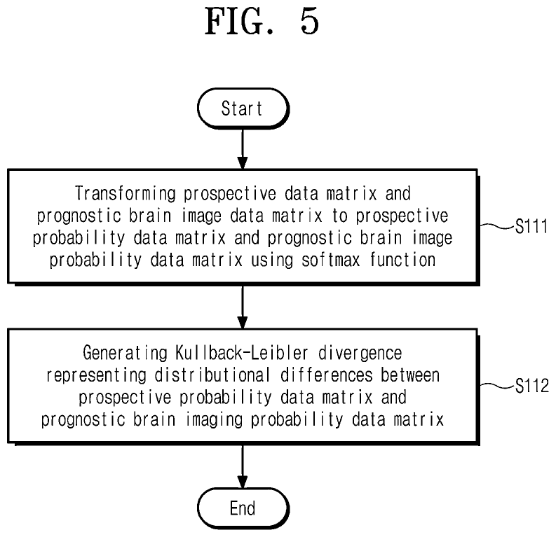

FIG. 5 is a flowchart showing the steps of generating a divergence function in step S110 of FIG. 4. Referring to FIGS. 1, 2, 4, and 5, the AI learning unit 110 uses a softmax function for the prospective data matrix and the prognostic brain image data matrix, respectively, to generate a prospective probability data matrix. and a prognostic brain image probability data matrix (S111); and generating a Kullback-Leibler divergence function representing the distribution difference between the prospective probability data matrix and the prognostic brain image probability data matrix (S112).

The AI learning unit 110 generates a first sub-objective function by applying a first combination coefficient to the cross-entropy loss function; generating a second sub-objective function by applying a second coupling coefficient to the divergence function; generating a normalization term based on the size of the projection matrix and the size of the coefficient vector to reduce the complexity of the projection matrix and coefficient vector; and generating an objective function based on the first sub-objective function, the second sub-objective function, and the normalization term.

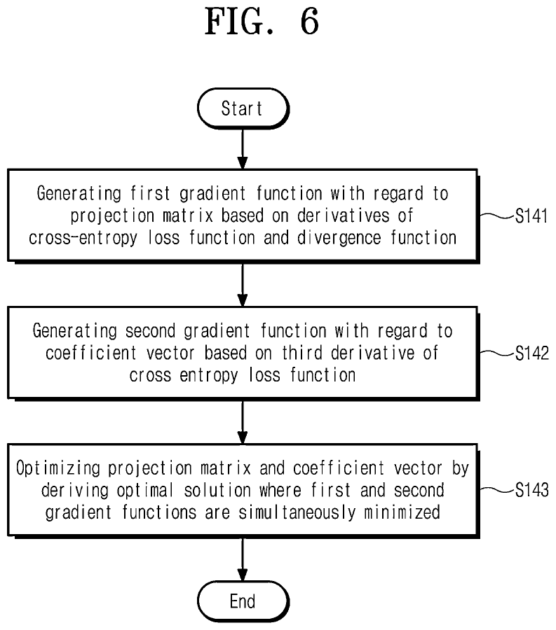

FIG. 6 is a flowchart specifically showing step S140 of FIG. 4. Referring to FIGS. 1, 2, 4, and 6, the AI learning unit 110 generates a first gradient function of the objective function with respect to the projection matrix based on the first derivative of the cross-entropy loss function with respect to the projection matrix and second derivative of divergence function with respect to the projection matrix (S141); generates a second gradient function of the objective function with respect to the coefficient vector based on a third derivative of the cross entropy loss function with respect to the coefficient vector (S142); optimizes the projection matrix and coefficient vector by deriving an optimal solution of the projection matrix and coefficient vector in which the sizes of the first gradient function and the second gradient function are simultaneously minimized (S143). In an embodiment, the first gradient function may include the first derivative, the second derivative, and a linear function of the projection matrix. The second gradient function may include the third derivative and a linear function of the coefficient vector.

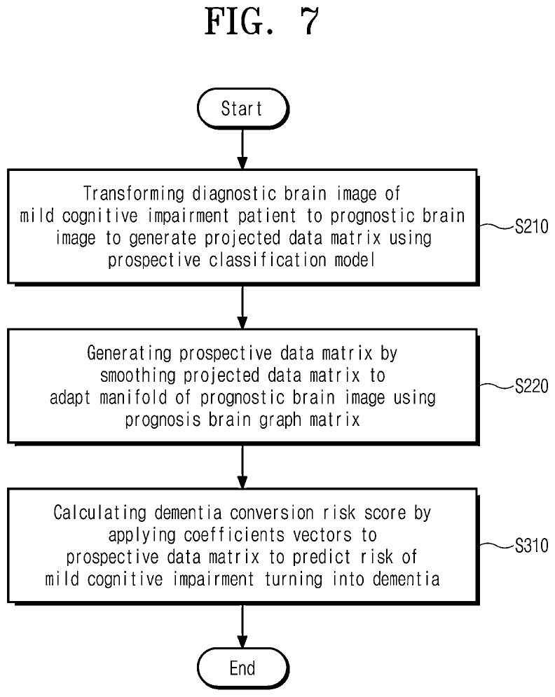

FIG. 7 is a flowchart specifically showing step S200 and step S300 of FIG. 2. Referring to FIGS. 1, 2, and 7, the feature transition unit 120 converts the diagnostic brain image data matrix acquired at the time of diagnosis of a patient with mild cognitive impairment by the learned (trained) projection matrix of the prospective classification model into a projection data matrix (S210). The feature transition unit 120 generates the prospective data matrix by smoothing the projection data matrix to adapt to the manifold of the prognostic brain image data matrix by the brain graph matrix of the prospective classification model (S220).

More specifically, the feature transition unit 120 converts the diagnostic brain image data matrix of a patient with mild cognitive impairment acquired at the time of diagnosis by the learned projection matrix of the prospective classification model to generate a projection data matrix. The feature transition unit 120 generates a brain graph matrix based on the correlation matrix representing the connection information of the feature regions of the brain image data matrix and the diagonal matrix of the correlation matrix. Ther feature transition unit 120 converts the projection data matrix to the prognostic brain image data matrix using the brain graph matrix of the prospective classification model to smooth the prospective data matrix and adapt to the manifold of the prognostic brain image data matrix.

The dementia risk prediction unit 130 predicts the risk of a mild cognitive impairment patient converting into a dementia patient by applying the learned coefficient vector of the prospective classification model to the prospective data matrix generated by the feature transition unit 120 and calculating the risk score indicating the probability that mild cognitive impairment converts to dementia (S310). Hereinafter, a prospective classification device for predicting dementia and its operating method according to an embodiment of the present invention will be described in more detail.

The operation method of the prospective classification device consists of three phases of preprocessing, projection and classification. In preprocessing, brain images are converted to brain volume features using voxel-based morphometry (VBM) analysis. Then, the projection that learns the transition from diagnosis to prognosis is performed, and the classification for AD conversion risk is conducted based on the transitioned features. The KL-divergence loss is computed in the projection step and the cross-entropy loss is measured in the classification step. Learning for the projection and learning for the classification are implemented as an end-to-end procedure by binding both losses into one objective function.

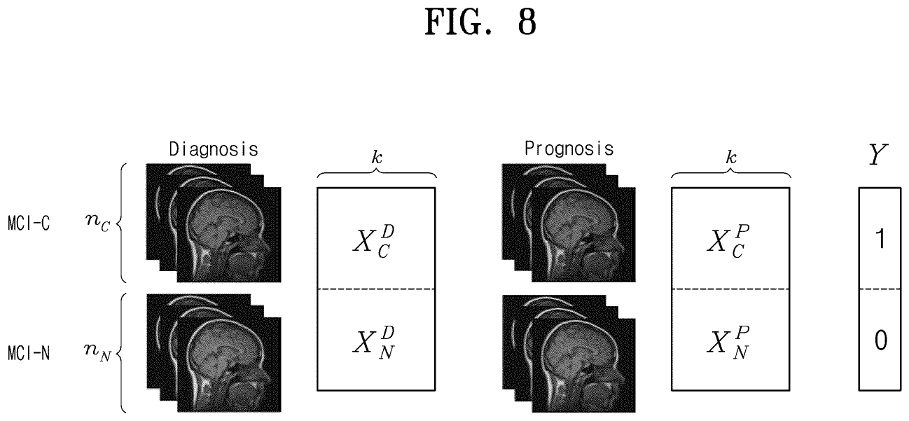

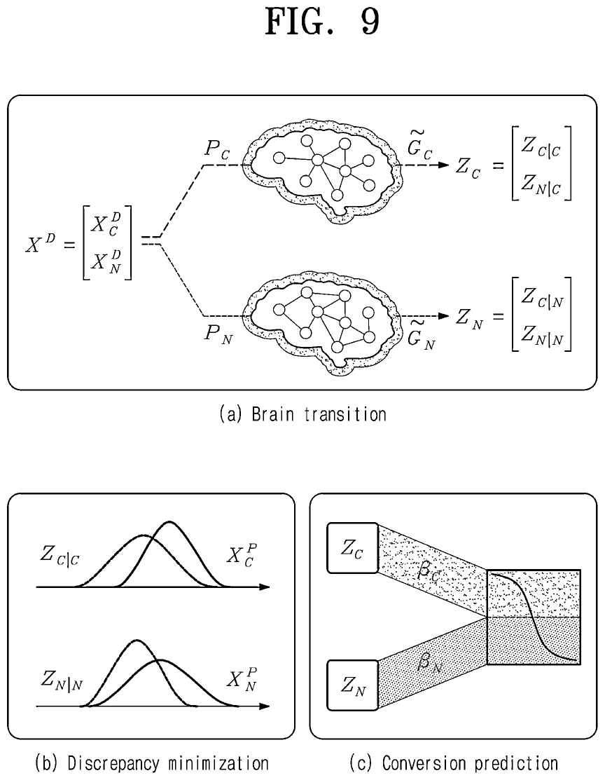

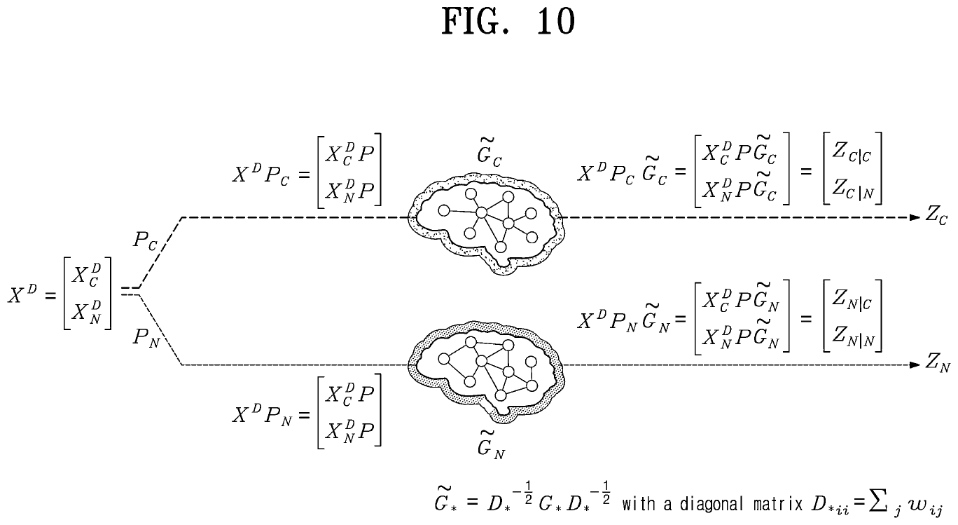

During brain transition, the brain volume features are first transformed through the projection matrix P that learns longitudinal changes between diagnosis and prognosis. The transformed features are then adapted to manifold of prognosis features. After the brain transition, the AD conversion risk is calculated using the transitioned and adapted features. For this purpose, any classifier can be used, but for convenience, the method of the present invention uses logistic regression to simultaneously obtain the information about the features primarily contribute to the discrimination problem of AD conversion/non-conversion. The values of coefficient β of logistic regression provide this information. The proposed method boils down to the problem of estimation of the projection matrix P and the coefficient vector β. FIG. 8 is an example diagram of diagnostic data and prognostic data to explain the operation method of the prospective classification device according to an embodiment of the present invention. FIGS. 9 and 10 are conceptual diagrams showing the process of converting diagnostic brain image data into prognostic brain image data according to an embodiment of the present invention. Let the data matrices of diagnosis and prognosis be denoted as X^{D} and prognosis X^{P}, respectively. To represent the prospective data matrix Z, the brain features of diagnosis are linearly transformed by projection matrix P and adapted by brain graph matrix G of prognosis.

$$Z = {X}^{D} P G \qquad ( 1 )$$

The projection matrix P is obtained by minimizing the actual values of prognosis features and the values of the projected features. The discrepancy can be measured using Kullback-Leibler (KL) divergence which measures the difference between two distributions. So each data matrices Z and X^{P} are converted to probability matrices P_{Z} (prospective probability data matrix) and P_{X}_{^{P}} (prognostic brain image probability data matrix) by using softmax function. The KL-divergence of two distributions (prospective probability data matrix and prognostic brain image probability data matrix) can be calculated as equation below.

$$K L ( Z \vert \vert {X}^{P} ) = {\sum }_{i = 1}^{n} {𝒫}_{{Z}^{( i )}} log ( \frac{{𝒫}_{{Z}^{( i )}}}{{𝒫}_{{X}_{( i )}^{P}}} ) . \qquad ( 2 )$$

The KL-divergence approaches 0, if two distributions are similar, it increases otherwise. On the other hand, the simple transformation X^{D} P provides only the longitudinal change of diagnosis-prognosis. Adaptation to the prognosis manifold may be considered to make the projected data more similar to the actual prognosis data. Manifold of data usually represented as graph. In the proposed method, the brain graph matrix G=(ROI, W) serves to smooth the projected data X^{D} P to adapt to the manifold of X^{P}. It provides the connectivity information between the ROI features of X^{P} and is constructed by a correlation matrix W={wᵢⱼ}. The higher the ROI correlation value between i and j, the stronger the wᵢⱼ. The brain graph matrix G is defined as the normalized graph,

$$G = {D}^{- \frac{1}{2}} W {D}^{- \frac{1}{2}}$$

with a diagonal matrix Dᵢᵢ=Σⱼwᵢⱼ.

The prospective transition can be useful for predicting when data sets from different points in time are provided. And furthermore, given the labels of the data, i.e., in the case of supervised learning, it is possible to determine the optimal P in the direction of reducing the loss, which is the difference between the label and the output, as well as the longitudinal difference and manifold adaptation. To classify the conversion from MCI to AD, a logistic classifier can be used. The converted features can be used as input for AD conversion risk as shown in the formula below. To classify MCI conversion to AD, logistic classifier is employed. The transitioned features are used as the input of AD conversion risk i as below:

$$\hat{Y} = \frac{1}{1 + {e}^{- Z β}} . \qquad ( 3 )$$

In general, the optimal β is obtained by least squares estimation. However, since Z in Equation (1) is not fixed, the least squares estimate of β cannot be applied. In fact, Z is determined by P and vice versa. This is because P is determined to reduce the loss between Y and Ŷ. In the proposed method, cross-entropy loss is used defined as:

$$L ( P , β ) = {Y}^{T} log \hat{Y} + {( 1 - Y )}^{T} log ( 1 - \hat{Y} ) . \qquad ( 4 )$$

To find P and β, the objective of prospective classification is designed as below by combining Equation (2) and (4), and regularizers.

$$arg {min}_{β , P} {γ}_{L} L ( P , β ) + {γ}_{K} K ( P ) + {‖ P ‖}_{2}^{2} + {‖ β ‖}_{2}^{2} \qquad ( 5 )$$

where ∥P∥₂² and ∥β∥₂² penalize the complexity, and γ_{L} and γ_{K} are combining coefficients (γ*≥0). The objective function is sum of a first sub-objective function with a first coupling coefficient applied to the cross-entropy loss function, a second sub-objective function with a second coupling coefficient applied to the divergence function, and normalization items (magnitude squared of the projection matrix ∥P∥₂², magnitude squared of the coefficient vector ∥β∥). The objective function is optimized by gradient descent method.

Minimization over P: To find gradient w.r.t the projection matrix P, the first and second terms in the objective function are derived. The derivative of regularizer term is negligible. The derivative of cross-entropy loss and the KL divergence is derived as follows:

$$\frac{\partial L ( P , β )}{\partial P} = {G}^{T} {X}^{{D}^{T}} ( \hat{Y} - Y ) {β}^{T} \qquad ( 6 )$$

$$\frac{\partial K ( P )}{\partial P} = {Σ}_{i = 1}^{n} [ ( Diag ( {𝒫}_{{Z}^{( i )}} ) - {𝒫}_{{Z}^{( i )}} {𝒫}_{{Z}^{( i )}}^{T} ) {G}^{T} {X}_{( i )}^{{D}^{T}} ( log {𝒫}_{{Z}^{( i )}} - log {𝒫}_{{X}_{( i )}^{P}} ) ]$$

$$As a result , the gradient of P is$$

$$∇ P = {γ}_{L} \frac{\partial L ( P , β )}{\partial P} + {γ}_{K} \frac{\partial K ( P )}{\partial P} + 2 P .$$

The first gradient function may include a first derivative of the projection matrix of the cross entropy loss function, a second derivative of the projection matrix of the divergence function, and a linear function of the projection matrix P. The initial P is set as the correlation matrix of (X^{D}, X^{P}).

Minimization over β: the derivative of coefficient vector β for the classifier is

$$\frac{\partial L ( P , β )}{\partial β} = {Z}^{T} ( \hat{Y} - Y ) ,$$

The gradient of β (second gradient function) including the regularizer is

$$∇ β = {γ}_{L} \frac{\partial L ( P , β )}{\partial β} + 2 β . \qquad ( 7 )$$

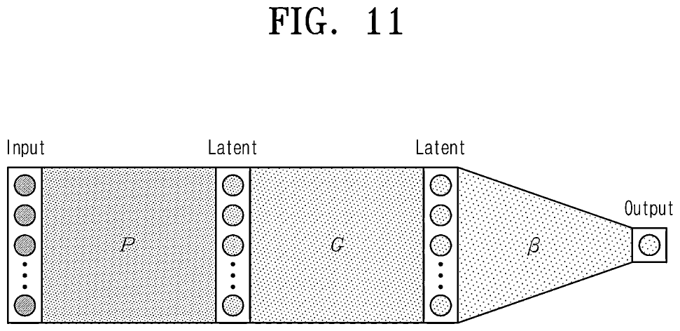

The second gradient function is sum of the third derivative of cross-entropy loss function with respect the coefficient vector and a linear function of coefficient vector. When the proposed model is trained on the preprocessed data, the KL-divergence loss and cross-entropy loss are minimized simultaneously, not as an end-to-end procedure. Optimizations for P and β seem complicated because they are intertwined. However, using the property that the two parameters are sequentially related, it is easy to obtain a solution with a structure similar to a multi-layer perceptron. By constructing a structure as shown in FIG. 11, the values of P and β are updated using the gradients in Equation (6) and (7) analogously to weight update of an ordinary neural network. In this way, the projection matrix and coefficient vector can be optimized by deriving an optimal solution of the projection matrix and coefficient vector that simultaneously minimizes the sizes of the first gradient function and the second gradient function.

The model for prospective classification is trained through the aforementioned processes. The model is trained to transform diagnosis MRIs to similar with prognosis MRIs, and then, is optimized to accurately predict the AD conversion for transitioned MRIs. After the model training and optimization, new MCI patient data can be applied to the model to predict AD conversion. In this case, only MRIs for the time of diagnosis is required for new data. The model transforms the input new MRI to the future. Then, the AD conversion risk is calculated for the transitioned virtual MRI. Therefore, the proposed method enables early diagnosis of AD conversion for MCI patients without directly confirming longitudinal change through follow-up.

The prospective classification model is learned through the process mentioned above. A prospective classification model is trained to transform diagnostic MRI to resemble prognostic MRI and is then optimized to accurately predict AD transformation for the converted MRI. After model training and optimization, new MCI patient data can be applied to the model to predict AD conversion. In this case, new data requires only the MRI at the time of diagnosis. The model converts the new MRI input into future data. Then, the AD conversion risk is calculated for the converted virtual MRI. Therefore, the operating method of the prospective classification device according to the embodiment of the present invention can diagnose AD conversion to MCI patients early without directly confirming longitudinal changes through follow-up observation.

FIG. 11 is a conceptual diagram of a prospective classification model according to an embodiment of the present invention. Brain metastases exhibit expected brain volume characteristics through linear transformation P from diagnosis to prognosis and smoothing of prognosis by brain graph G. AD conversion risk is predicted by a logistic classifier by estimating the coefficient vector β based on the converted features. Optimization for P and is solved by constructing a structure similar to an MLP network with four layers: input (X^{D})−latent(X^{P})−latent(X^{P})−output(Ŷ). The weight matrices of X^{D}−X^{P}, X^{P}−X^{P}, and X^{P}−Ŷ correspond to P, G, and β, respectively. The brain graph matrix G is not a parameter to learn but fixed by using brain graph adjacency matrix. And β provides information on key features contributing to the risk of AD conversion/non-conversion.

Hereinafter, an experiment to verify the performance of the prospective classification device and operation method according to an embodiment of the present invention is described. The data used in the experiments of the present invention were obtained from the ADNI (Alzheimer's Disease Neuroimaging Initiative) database. Subjects with more than one record were labeled according to the criteria that are generally used to divide AD and MCI subjects as follows. (a) MCI subjects: MMSE scores between 24-30, a memory complaint, having objective memory loss measured by education adjusted scores on Wechsler Memory Scale Logical Memory II, a Clinical Dementia Rating (CDR) of 0.5. (b) AD subjects: MMSE scores between 20-26, CDR of 0.5 or 1.0. As a result, 147 subjects including 73 MCI-C and 74 MCI-N were selected, and Table 1 provides a summary.

TABLE 1
Demographics of subjects in the dataset.

| Category | | MCI-C (n = 74) | MCI-N (n = 73) |
|---|---|---|---|
| Age at baseline | | 74.6 ± 7.4 | 75.7 ± 5.7 |
| Follow-up duration (months) | | 28.4 ± 10.1 | 27.0 ± 9.2 |
| MMSE | baseline | 27.4 ± 1.6 | 26.8 ± 1.5 |
| | follow-up | 24.3 ± 4.8 | 24.3 ± 3.1 |
| CDR | baseline | 0/147/0 | 1/29/0 |
| (0/0.5/1) | follow-up | 0/87/51 | 0/22/8 |

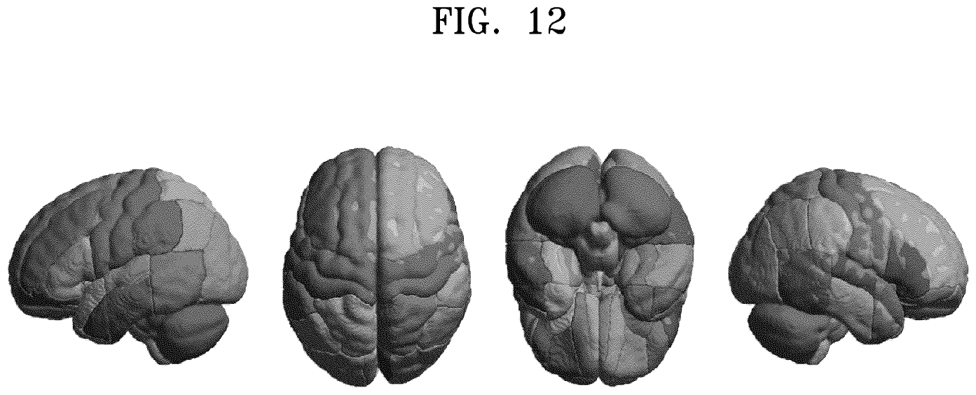

For the selected subjects, a total of 294 MR images for each diagnosis and prognosis were collected. MR images were preprocessed to extract ROI-based features. First, anterior commissure (AC)-posterior commissure (PC) correction was done on all images. Then, the computational anatomy toolbox (CAT12: http://www.neuro.uni-jena.de/cat/) for SPM package is used to segment structural MR images into three different tissues: gray matter (GM), white matter (WM), and cerebrospinal fluid (CSF). For a given brain image with three segmented tissues, the subject-labeled images were obtained based on the Hammers atlas, which consists of 95 ROIs as shown in FIG. 12 by the BrainNet Viewer. Finally, the volumes of GM tissue of the 95 ROIs were used, which were normalized by the total intracranial volume estimated by the summation of GM, WM, and CSF volumes from all ROIs, as features for a given subject.

In this subsection, it will be described the results for one-way domain adaptation. The proposed method has two objectives to reduce the discrepancy between distributions of brain volume data (distribution matching) and to make the relationship between brain ROIs in the two domains similar (connectivity mapping). The results for each objective were represented in order.

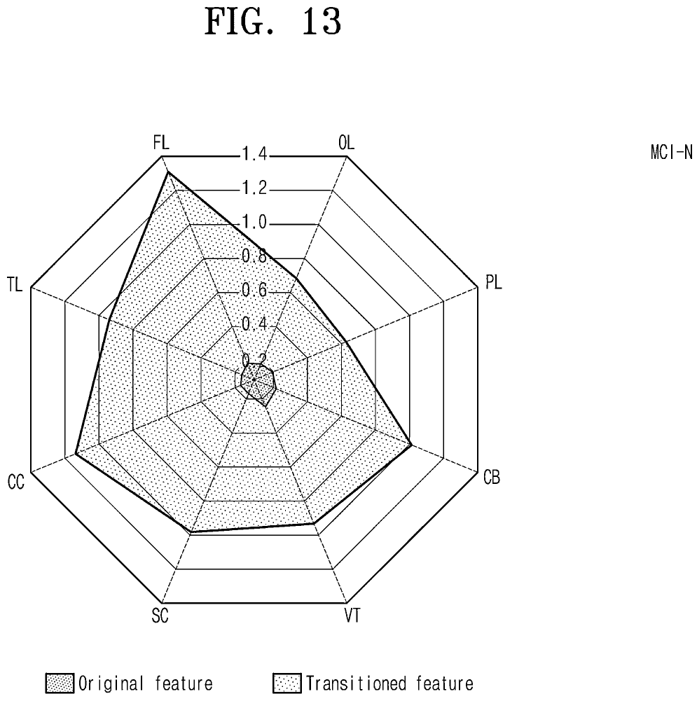

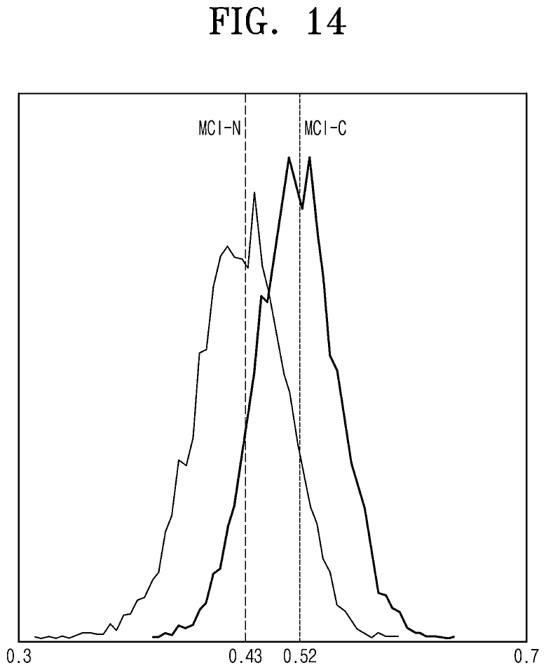

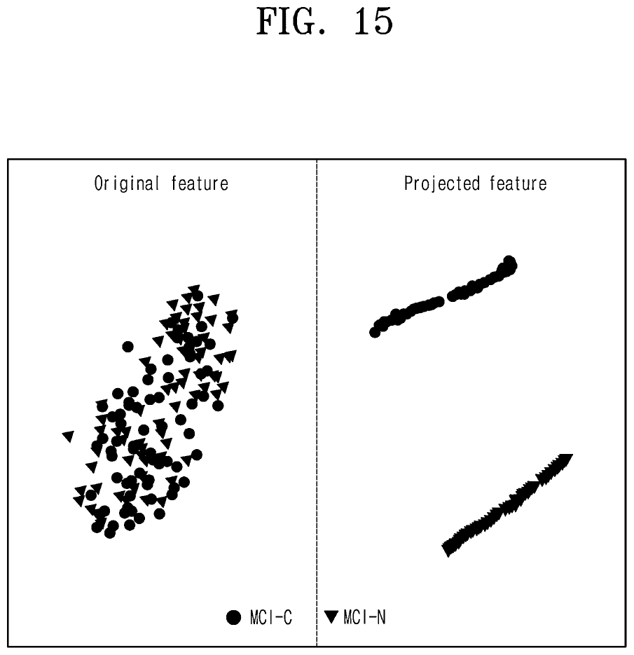

The results for prospective classification are described in FIGS. 13 to 15. FIG. 13 depicts the effect of the transition of brain ROIs by the projection matrix P. The eight axes included in the radial diagram represent the upper categories from 95 ROIs. The blue area represents the difference between MCI-C and MCI-N before the transition, and the red area is for that of after the transition. The change shows that the transition from diagnosis to prognosis makes the feature space more clearly distinguish the patterns of two groups. This change means that the transition from diagnosis to prognosis creates a feature space that can more clearly distinguish the patterns of the two groups. It also provides more separable outcome in terms of predicted score distributions for AD transition risk as shown in FIG. 14.

First, FIG. 13 shows the effect of brain transition where 95 ROIs are divided into 8 upper categories: frontal lobe (FL), occipital lobe (OL), parietal lobe (PL), cerebellum (CB), ventricle (VT), subcortical (SC), cingulate cortex (CC), and temporal lobe (TL). The blue colored area indicates the difference of original features in each category between MCI-C and MCI-N, and the red colored area is that of transitioned features. As a result, the two groups are clearly distinct after the transition, whereas before the transition they overlap and are difficult to distinguish. This implies that the transitioned ROIs is more discriminating than the ROIs at diagnosis, which means that the projection matrix P has successfully learned the brain ROI feature representation that transitions the two groups from diagnosis to prognosis. Accordingly, the distributions of AD conversion risk score V are well differentiated as shown in FIG. 14.

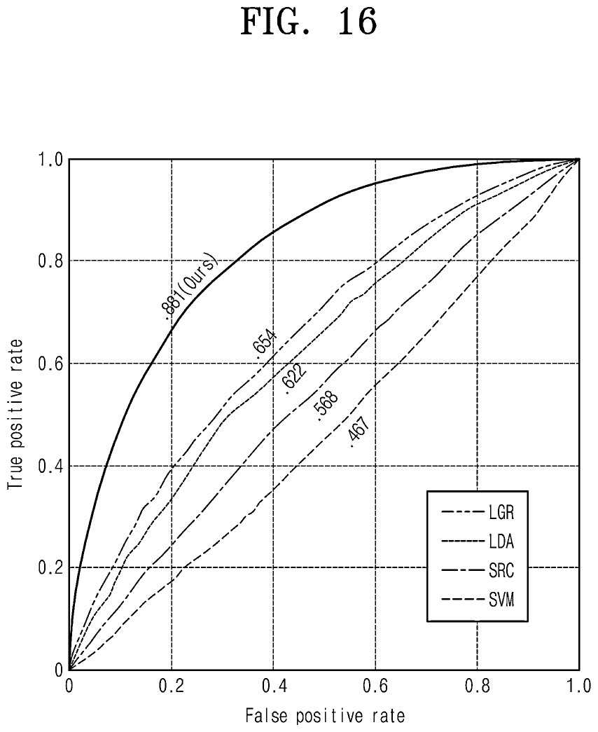

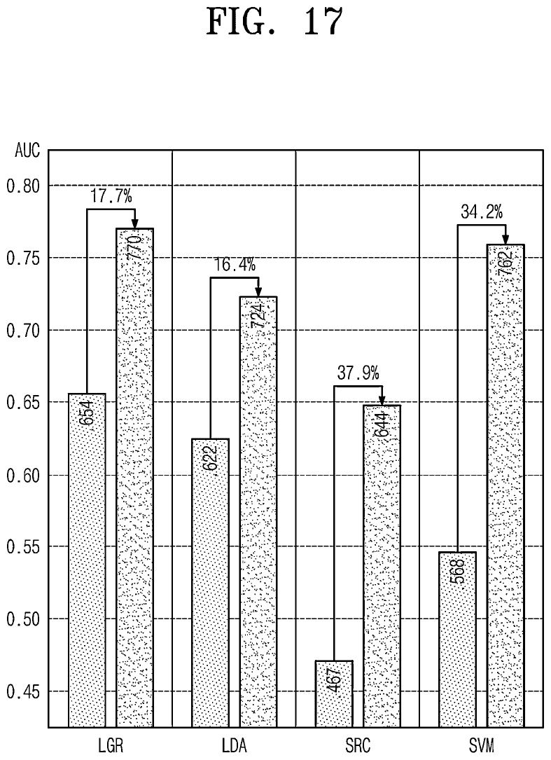

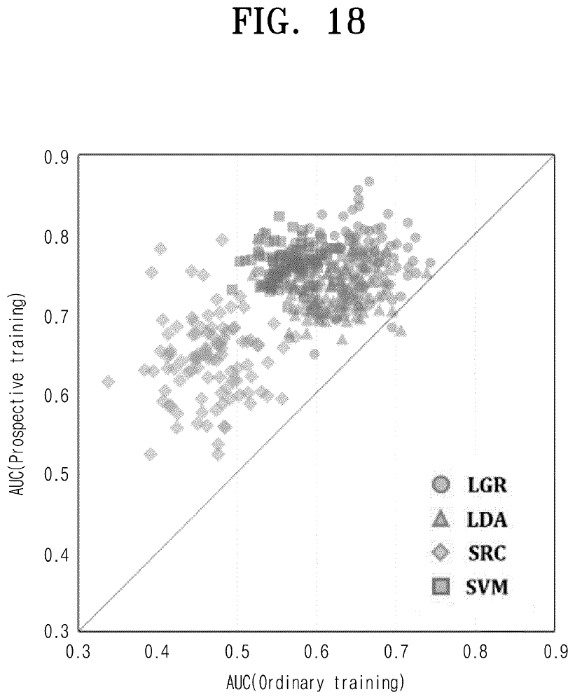

To compare the accuracy of AD conversion prediction, we designed experiments to measure the effect of the transitioned features with four competing algorithms: LGR (logistic regression), LDA (linear discriminant analysis), SRC (sparse representation classifier), and SVM (support vector machine). First, those algorithms predicted the AD conversion of MCI patients by directly training the diagnosis data (ordinary training). Second, the prediction was performed by training the transitioned features via the proposed method (prospective training). The entire experiments were repeated 100 times of 5-fold cross validation, and the prediction accuracy was measured with the area under receiving operating characteristic curve (AUC). The overall comparison results are shown in FIGS. 16 to 18. FIG. 16 presents the overall AUC comparison results using the respective ROC curves. FIG. 17 indicates the overall performance improvement by using the transitioned features. FIG. 18 of the scatter plot depicts the individual results of comparing AUCs with/without transitioned features. A point located above the diagonal line performs better than the algorithm on the vertical axis.

FIG. 16 shows the ROC curves of the five algorithms including the proposed method. The AUC of our method is 0.881, indicating 34.7% up to 88.7% higher performance than the competing algorithms. FIGS. 17 and 18 compare the results of AD conversion prediction by competing algorithms (LGR, LDA, SRC, and SVM) with the ordinary training and the prospective training. FIG. 17 provides the overall performance improvement by the effect of transition: the transitioned features were fed to input of LGR, LDA, SRC, and SVM. The AUC of each of algorithms was improved about 26.5% on average. FIG. 18 represents the individual performance improvement by the prospective training in each repeated experiment. A point in the scatter plot located above the diagonal line means that the algorithm on the vertical axis performs better. FIG. 18 shows that most of the dots lie above the diagonal line. Among the algorithms used as comparison methods, the remaining three, except for LGR, were also used to predict AD conversion in previous studies. Coupe et al used changes in hippocampal volume and MMSE as criteria for AD conversion and predicted them through LDA, and derived an AUC performance of 0.657 on average. Xu et al performed SRC-based prediction using MRI and PET as multimodal data, and the performance for MRI data was 0.506. Wei et al applied SVM with MRI variables selected through feature selection and derived an average performance of 0.742. Note that 0.881 AUC obtained by our method outperformed the existing studies. FIGS. 17 and 18 experimentally prove that the transitioned features are useful not only for the logistic classifier, but also for other algorithms.

The proposed method was also compared with CNN of the deep learning-based method. This analysis purpose not only to simply compare the prediction accuracy but also to empirically identify the change of discriminative power of methods according to the number of training samples. For this, the comparative experiments were performed on datasets reduced by 50% and 20% compared to the original dataset. Among various CNN models, ResNet was selected as a comparison method to circumvent the vanishing gradient problem. ResNet was trained on the voxel level 3-D images data without preprocessing. The model architecture designed as the 18-depth network with a total of 71 layers including 78 connections, 20 convolutional layers, and a fully connected layer. The model parameters were optimized by the stochastic gradient descent method with 0.001 of learning rate. The model performance was measured by the classification accuracy for 100 times repeated experiments.

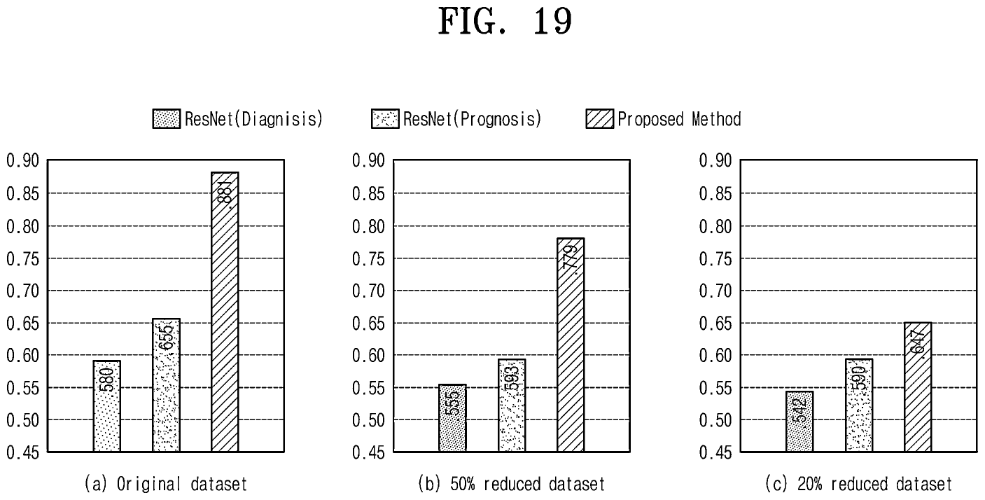

The results for comparison analysis with CNN are shown in FIG. 19 where the green and blue bars denote the classification accuracy of ResNet trained the diagnosis and prognosis data, respectively, and the red bars are the results of the proposed method. At first, in case of training the original dataset as shown in FIG. 19 (a), the proposed method performed 44.1% and 27.6% better than ResNet trained the diagnosis and prognosis data, respectively. The higher accuracy of the proposed method was also presented in 50% and 20% reduced datasets as shown in FIG. 19 (b) and (c). In particular, the accuracy of the proposed method in 20% reduced dataset was almost same with the accuracy of ResNet trained the original prognosis dataset. Despite the superiority of ResNet, it requires a sufficient amount of data to train a large number of parameters. However, in reality, collecting large amounts of data is not easy because years or decades of follow-up are required to collect cases of conversion from MCI to AD. This is a frequent difficulty in medical domain applications as exemplified in this study. Therefore, lightweight and accurate models that can be trained with small amounts of data are inevitable. Comparison with ResNet shows that the proposed method saves more data and shows better performance when the data is limited. Considering these points, the proposed method has advantages and leaves the possibility of extension for similar cases.

Coefficient β in the logistic regression by Equation (3) tells which regions of the brain play an important role of progression to AD conversion: higher β values indicate more important regions.

According to an embodiment of the present invention, the characteristics of diagnostic brain imaging data obtained at the time of diagnosis of a patient with mild cognitive impairment are converted into features of prognostic brain imaging data corresponding to the prognostic time after the diagnosis time to treat mild cognitive impairment.

According to an embodiment of the present invention, the features of brain imaging data at a recent time corresponding to the prognostic time are converted into features of past diagnostic brain imaging data of longitudinal data sequentially collected according to a time series. By matching the attributes, the possibility of converting a patient with mild cognitive impairment into a dementia patient can be accurately predicted by a prospective domain adaptation algorithm.

As described above, in the method according to an embodiment of the present invention, prospective classification is performed to predict conversion from MCI to AD. The method according to an embodiment of the present invention performs classification using a brain image converted to the time of prognosis, rather than the current image at the time of diagnosis. This process includes adaptation to various prognostic features. The risk of AD conversion after brain metastases is calculated using a logistic classifier that simultaneously provides information about the primary brain region, which contributes to the problem of distinguishing AD conversion/non-conversion. Experimental results on the ADNI data set show that the conversion and non-conversion groups are more distinct after conversion. It also shows that the converted features can be used in any classifier and improve the original performance.

The above-described methods may be embodied in the form of processor executing program instructions recorded in computer-readable medium by various computer means. The computer readable medium may record program instructions, data files, data structures, and the like, alone or in combination. Program instructions recorded on the media may be those specially designed and constructed for the purposes of the inventive concept, or they may be of the kind well-known and available to those having skill in the computer software arts. Examples of computer readable recording media include magnetic media such as hard disks, floppy disks and magnetic tape, optical media such as CD-ROMs, DVDs, and magnetic disks such as floppy disks, Magneto-optical media, and hardware devices specifically configured to store and execute program instructions, such as ROM, RAM, flash memory, and the like. Examples of program instructions include not only machine code generated by a compiler, but also high-level language code that can be executed by a computer using an interpreter or the like. The hardware device described above may be configured to operate as one or more software modules to perform the operations of the present disclosure, and vice versa.

Although the embodiments have been described by the limited embodiments and the drawings as described above, various modifications and variations are possible to those skilled in the art from the above description. For example, the described techniques may be performed in a different order than the described method, and/or components of the described systems, structures, devices, circuits, etc. may be combined or combined in a different form than the described method, or other components, or even when replaced or substituted by equivalents, an appropriate result can be achieved. Therefore, other implementations, other embodiments, and equivalents of the claims also fall within the scope of the claims described below. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A prospective classification device for predicting dementia comprising:
at least one processor configured to predict a risk of a mild cognitive impairment patient being converted to a dementia patient by executing a prospective classification program recorded in memory,
wherein the at least one processor is configured to:
convert features of diagnostic brain imaging data of the patient with mild cognitive impairment obtained at the time of diagnosis into features of prognostic brain imaging data corresponding to a prognostic time after the time of diagnosis by a prospective classification model; and
predict the risk of the mild cognitive impairment patient being converted into the dementia patient based on the features of the prognostic brain imaging data converted from the features of the diagnostic brain imaging data,
wherein the prospective classification model is a model trained to transform features of diagnostic brain imaging data acquired for patients suffering from mild cognitive impairment at a first time point to prognostic brain imaging data acquired at a second time point for the patients after the first time point.

2. The prospective classification device of claim 1,
wherein the at least one processor is configured to:
convert a diagnostic brain image data matrix obtained at the time of diagnosis of the patient with mild cognitive impairment to generate a projection data matrix by a projection matrix of the trained prospective classification model;
smooth the projection data matrix to adapt to a manifold of prognostic brain image data matrix to generate a prospective data matrix by a brain graph matrix of the trained prospective classification model; and
predict the risk the patient with mild cognitive impairment being converted to a dementia patient by calculating a dementia conversion risk score indicating a probability that mild cognitive impairment being converted to dementia, the dementia conversion risk score being calculated by applying a coefficient vector of the prospective classification model to the prospective data matrix.

3. The prospective classification device of claim 2,
wherein the at least one processor is configured to:
generate the brain graph matrix based on a correlation matrix representing connection information of feature regions of the prognostic brain image data matrix, and a diagonal matrix of the correlation matrix.

4. The prospective classification device of claim 1,
wherein the at least one processor is configured to:
convert a diagnostic brain image data matrix of each patient suffering from mild cognitive impairment by a projection matrix to generate a projection data matrix;
calculate a brain graph matrix representing a manifold of prognostic brain image data matrix for each of the first patients who suffered from mild cognitive impairment and then converted to dementia and the second patients not converted to dementia after suffering from mild cognitive impairment;
generate a prospective data matrix by smoothing the projection data matrix of each of the first patients and the second patients to adapted to the manifold of the prognostic brain image data matrix using the brain graph matrix;
generate a divergence function representing a distribution difference between the prospective data matrix generated for each of the first patients and the second patients and the prognostic brain image data matrix of the patient corresponding to each prospective data matrix;
calculate a dementia conversion risk score indicating a probability that mild cognitive impairment converts to dementia by variables including the prospective data matrix and coefficient vector for each of the first patients and the second patients;
generate a cross-entropy loss function between the dementia conversion risk score calculated for each of the first patients and the second patients and the dementia conversion correct answer labels of the first patients and the second patients; and
optimize the projection matrix and the coefficient vector based on a derivative generated by partial differentiation of an objective function defined by the divergence function and the cross-entropy loss function with respect to the projection matrix and the coefficient vector.

5. The prospective classification device of claim 4,
wherein the at least one processor is configured to:
calculate a first gradient function of the objective function with respect to the projection matrix based on a first derivative of the cross entropy loss function with respect to the projection matrix and a second derivative of the divergence function with respect to the projection matrix;
calculate a second gradient function for the coefficient vector of the objective function based on a third derivative of the cross-entropy loss function with respect to the coefficient vector; and
optimize the projection matrix and the coefficient vector by deriving an optimal solution of the projection matrix and the coefficient vector in which the magnitudes of the first gradient function and the second gradient function are simultaneously minimized.

6. The prospective classification device of claim 5,
wherein the first gradient function includes the first derivative, the second derivative, and the linear function of the projection matrix, and the second gradient function includes the third derivative and the linear function of the coefficient vector.

7. The prospective classification device of claim 4,
wherein the at least one processor is configured to:
convert the prospective data matrix and the prognostic brain image data matrix into a prospective probability data matrix and a prognostic brain image probability data matrix, respectively, using a softmax function; and
generate a Kullback-Leibler divergence function representing the distribution difference between the prospective probability data matrix and the prognostic brain image probability data matrix.

8. The prospective classification device of claim 4,
wherein the at least one processor is configured to:
generate a first sub-objective function by applying a first combination coefficient to the cross-entropy loss function;
generate a second sub-objective function by applying a second coupling coefficient to the divergence function;
generate a normalization term based on a size of the projection matrix and a size of the coefficient vector to reduce the complexity of the projection matrix and the coefficient vector; and
generate the objective function based on the first sub-objective function, the second sub-objective function, and the normalization term.

9. An operation method of prospective classification device for predicting dementia comprising:
predicting a risk of a mild cognitive impairment patient being converted to a dementia patient by executing a prospective classification program recorded in memory by at least one processor,
wherein the predicting of the risk comprises:
converting features of diagnostic brain imaging data of the patient with mild cognitive impairment obtained at the time of diagnosis into features of prognostic brain imaging data corresponding to a prognostic time after the time of diagnosis by a prospective classification model; and
predicting the risk of the mild cognitive impairment patient being converted into the dementia patient based on the features of the prognostic brain imaging data converted from the features of the diagnostic brain imaging data,
wherein the prospective classification model is a model trained to transform features of diagnostic brain imaging data acquired for patients suffering from mild cognitive impairment at a first time point to prognostic brain imaging data acquired at a second time point for the patients after the first time point.

10. The operation method of claim 9,
wherein converting of features of the diagnostic brain imaging data comprises:
converting a diagnostic brain image data matrix obtained at the time of diagnosis of the patient with mild cognitive impairment to generate a projection data matrix by a projection matrix of the trained prospective classification model;
smoothing the projection data matrix to adapt to a manifold of prognostic brain imaging data matrix to generate a prospective data matrix by a brain graph matrix of the trained prospective classification model; and
predicting the risk the patient with mild cognitive impairment being converted to a dementia patient by calculating a dementia conversion risk score indicating a probability that mild cognitive impairment being converted to dementia, the dementia conversion risk score being calculated by applying a coefficient vector of the prospective classification model to the prospective data matrix.

11. The operation method of claim 10,
wherein converting of features of the diagnostic brain imaging data further comprises:
generating the brain graph matrix based on a correlation matrix representing connection information of feature regions of the prognostic brain image data matrix, and a diagonal matrix of the correlation matrix.

12. The operation method of claim 9, further comprising:
learning the prospective classification model based on the diagnostic brain imaging data and the prognostic brain imaging data, by the at least one processor,
wherein the learning of the prospective classification model comprises:
converting diagnostic brain image data matrix of each patient suffering from mild cognitive impairment by a projection matrix to generate a projection data matrix;
calculating a brain graph matrix representing a manifold of prognostic brain image data matrix for each of the first patients who suffered from mild cognitive impairment and then converted to dementia and the second patients not converted to dementia after suffering from mild cognitive impairment;
generating a prospective data matrix by smoothing the projection data matrix of each of the first patients and the second patients to adapted to the manifold of the prognostic brain image data matrix using the brain graph matrix;
generating a divergence function representing a distribution difference between the prospective data matrix generated for each of the first patients and the second patients and the prognostic brain image data matrix of the patient corresponding to each prospective data matrix;
calculating a dementia conversion risk score indicating a probability that mild cognitive impairment converts to dementia by variables including the prospective data matrix and coefficient vector for each of the first patients and the second patients;
generating a cross-entropy loss function between the dementia conversion risk score calculated for each of the first patients and the second patients and the dementia conversion correct answer labels of the first patients and the second patients; and
optimizing the projection matrix and the coefficient vector based on a derivative generated by partial differentiation of an objective function defined by the divergence function and the cross-entropy loss function with respect to the projection matrix and the coefficient vector.

13. The operation method of claim 12,
wherein the optimizing of the projection matrix and the coefficient vector comprises:
calculating a first gradient function of the objective function with respect to the projection matrix based on a first derivative of the cross entropy loss function with respect to the projection matrix and a second derivative of the divergence function with respect to the projection matrix;
calculating a second gradient function for the coefficient vector of the objective function based on a third derivative of the cross-entropy loss function with respect to the coefficient vector; and
optimizing the projection matrix and the coefficient vector by deriving an optimal solution of the projection matrix and the coefficient vector in which the magnitudes of the first gradient function and the second gradient function are simultaneously minimized.

14. The operation method of claim 13, wherein the first gradient function includes the first derivative, the second derivative, and the linear function of the projection matrix, and the second gradient function includes the third derivative and the linear function of the coefficient vector.

15. The operation method of claim 12,
wherein the generating of the divergence function comprises:
converting the prospective data matrix and the prognostic brain image data matrix into a prospective probability data matrix and a prognostic brain image probability data matrix, respectively, using a softmax function; and
generating a Kullback-Leibler divergence function representing the distribution difference between the prospective probability data matrix and the prognostic brain image probability data matrix.

16. A prospective classification device of claim 12,
wherein the learning of the prospective classification model comprises:
generating a first sub-objective function by applying a first combination coefficient to the cross-entropy loss function;
generating a second sub-objective function by applying a second coupling coefficient to the divergence function;
generating a normalization term based on a size of the projection matrix and a size of the coefficient vector to reduce the complexity of the projection matrix and the coefficient vector; and
generating the objective function based on the first sub-objective function, the second sub-objective function, and the normalization term.

17. A computer-readable non-transitory recording medium on which a computer program for executing an operation method of a prospective classification device for predicting dementia according to claim 9 is recorded.
